(19) United States
(12) Patent Application Publication
(10) Pub. No.: US 2024/0367175 A1
(43) Pub. Date: Nov. 7, 2024

(54) HOLDER FOR AN INDIVIDUAL CAPILLARY TUBE, AND COLLECTING DEVICE FOR A PLURALITY OF HOLDERS

(71) Applicant: Alfred-Wegener-Institut, Helmholtz-Zentrum fuer Polar- und Meeresforschung, Bremerhaven (DE)

(72) Inventors: Johannes LEMBURG, Salem (DE); Christian BOCK, Bremen (DE); Rolf WITTIG, Bremerhaven (DE); Anette TILLMANN, Beverstedt (DE)

(21) Appl. No.: 18/683,778

(22) PCT Filed: Aug. 16, 2022
(86) PCT No.: PCT/DE2022/100602

§ 371 Date: Feb. 15, 2024

(30) Foreign Application Priority Data
Aug. 31, 2021 (DE) 10 2021 122 449.7

(51) Int. Cl. B01L 3/00; G09F 3/00

(52) U.S. Cl. CPC B01L 3/5453; G09F 3/0295; B01L 2300/0838

(57) ABSTRACT

A holder for a single capillary tube includes: an elongated carrier which at one end thereof has two clamping jaws at a distance from each other for receiving one end of the capillary tube and an other end thereof a handle with an elongated planar area, the handle being connected to the elongated carrier in one piece. The elongated carrier includes a planar area and is thinner than a diameter of a capillary rod to be held, and at one end the elongated carrier has an elongated slit forming two opposite arms including clamping jaws. A planar area of the handle includes a marking field for applying information. The marking field is arranged on one side or on both sides of the elongated carrier.

### CROSS-REFERENCE TO PRIOR APPLICATIONS

This application is a U.S. National Phase application under 35 U.S.C. § 371 of International Application No. PCT/DE2022/100602, filed on Aug. 16, 2022, and claims benefit to German Patent Application No. DE 10 2021 122 449.7, filed on Aug. 31, 2021. The International Application was published in German on Mar. 9, 2023 as WO/2023/030577 A1 under PCT Article 21 (2).

### FIELD

The invention relates to a holder for an individual capillary tube comprising an elongated carrier, which at one end has two clamping jaws at a distance from each other for receiving one end of the capillary tube, and at its other end a handle with an elongated, planar surface, wherein the handle is connected in one piece with the carrier, and also to a collecting device for a plurality of holders.

### BACKGROUND

Capillary tubes are, amongst other things, used for receiving samples, for example, blood samples or other aqueous samples, which in the transparent capillary tube are metrologically analysed in a repeated and destruction-free manner, for example in a spectrometer. Such tests are used, for example, in biochemical, pharmaceutical and medical analysis. The capillary tubes are most made of glass, have a diameter of between 1 mm and 1.8 mm and are very prone to breakage. They are therefore often handled with special closure caps, tongs or holders. It is important to clearly mark the filled capillary tubes, wherein this marking should be simple to assign and legible.

The prior art closest to the invention can be seen in DE 28 13 680 B1. Disclosed is a holder for an individual capillary tube for receiving a blood sample. The holder comprises an elongated carrier made of hard plastic which is connected in one piece to a handle. The latter can comprise four wings arranged in the shape of a cross so that it is centred well in a cylindrical sample container. The carrier has two clamping jaws opposite each other for receiving the capillary tube.

Known from DE 103 43 405 A1 is a holder for a single capillary tube for examination of the contents in an NMR spectrometer. The capillary tube is inserted into a rotationally symmetrical sample sleeve, the circular surface of which can be inscribed. The sample sleeve has two opposite centring arms which centre the capillary tube. The sample sleeve also has grooves on its outer side into which a gripping device for transporting the capillary tube can engage. Also disclosed is a container for receiving a plurality of sample sleeves with capillary tubes, wherein the sample sleeves are arranged in rows and columns in such a way that they can be gripped with the centring arms. Known from DE 101 11 674 A1 is a rotationally symmetrical fastening sleeve for a single capillary tube together with which it can be introduced into a so-called “spinner” (rotor of an air turbine for rotating the capillary tube) of an NRM spectrometer and centred. For transporting, the capillary tube can be closed with a sample lid which is suitable for the application of a DOT code to identify the measuring sample.

From US 2017/0087547 A1, a multiple-part sample container is known, in which a single glass tube is centred in a fluid-filled space. The glass rube is held with a handle and introduced into the cylindrical space. The handle is then broken off so that removal has to take place in another way. Marking surfaces are not provided. For centring the glass tube in the cylindrical space, it has two opposite sprung projections. Shown in design US 350 195 S, is a holder which holds two capillary tubes aligned at an angle with regard to each other. These are evidently open at the top. The holder also comprises two handles that are applied orthogonally with regard to the main plane and are curved and have no marking surfaces. The holder is not rotationally symmetrically and is solely intended for transporting both capillary tubes.

EP 2 848 309 A1 discloses a carrier for the simultaneous receiving of a plurality of capillary tubes, wherein these are marked with numbers. All the capillary tubes can be moved over a microtitre plate and emptied at the same time. Known from DE 10 2011 054 998 A1 is a container in which a plurality of capillary tubes can be stored and transported. The capillary tubes are fixed between clamping jaws and marked with numbers.

Known from EP 1 695 762 A2 is also a carrier for a plurality of capillary tubes, wherein the carrier is designed monolithically and for each capillary tube to be held has two, integrated and opposite clamping jaws. In the vicinity of these, the carrier is considerably thicker than the capillary tubes to be held, so that they are only held through clamping. Vertical positioning of the carrier takes place by way of a block-like rail opposite the clamping jaws, with which the carrier with all the inserted capillary tubes can be simultaneously placed in a machine. Handles are provided on both side of the rail as an insertion and locking aid. Direct marking of the tubes in the area of the rail is not provided. From WO 2009/026055 A1, a device is known with which a plurality of capillary tubes, which are arranged in rows and columns, are aligned radially and vertically by way of special active surfaces and can be inserted into an analysis apparatus. Rotational alignment of the individual capillary tubes about their own axis is not envisaged. At their lower end, the holders from the capillary tubes are closed, so that the capillary tubes project out of the holders with their full length and can be damaged. Individual marking of the individual capillary tubes is not possible.

### SUMMARY

In an embodiment, the present invention provides a holder for a single capillary tube, comprising: an elongated carrier which at one end thereof has two clamping jaws at a distance from each other configured to receive one end of the capillary tube and an other end thereof a handle with an elongated planar area, the handle being connected to the elongated carrier in one piece, wherein the elongated carrier comprises a planar area and is thinner than a diameter of a capillary tube to be held, and at one end the elongated carrier has an elongated slit forming two opposite arms comprising clamping jaws, wherein a planar area of the handle comprises a marking field configured for applying information, and wherein the marking field is arranged on one side or on both sides of the elongated carrier.

### BRIEF DESCRIPTION OF THE DRAWINGS

The present invention will be described in even greater detail below based on the exemplary figures. The invention is not limited to the exemplary embodiments. Other features and advantages of various embodiments of the present invention will become apparent by reading the following detailed description with reference to the attached drawings which illustrate the following:

FIG. 1 shows a view of the holder from above,

FIG. 2 shows a cross-section of the holder according to FIG. 1,

FIG. 3 shows the holder with stuck on inscription,

FIG. 4 shows the holder with capillary tubes in a spinner,

FIG. 5 shows a cross-section of the holder according to FIG. 4, and

FIG. 6 shows a collection device for a plurality of holders.

### DETAILED DESCRIPTION

In an embodiment, the present invention provides a holder of the type in question described in the introduction for an individual capillary tube in such a way, that particularly simple marking, handling, including transporting, use and storage with easily visible identification, as well as production, can be achieved. To solve this task, in accordance with the invention the carrier is solely designed as a planar area and thinner than the diameter of the capillary tube to be held, and, at its one end has an elongated slit, forming two opposite arms, wherein these arms form the clamping jaws, and in that the planar surface of the handle is designed as a marking field for attaching information, wherein the marking field is arranged on one side or both sides of the carrier.

In the invention, the holder is solely designed as a planar area and can also be called a “tag” (holder with capillary tube—“tubetag”). This allows the clamping jaws to be integrated into the planar area. This takes place through the provision of the elongated slit which divides one end of the planar area into two opposite arms. These then form the clamping jaws. The planar, thin design of the holder according to the invention, also provides good opportunities for gripping the holder in the area of the handle and adding information on a specially provided marking field, wherein its area is designed to be so large that clear, direct information about the contents of an individually held capillary tube can be provided, for example, pre-printed labels with an inscription or barcode. In this way, assignment of the marking to the individual capillary tube is clear. According to the invention, the marking field can be provided on one or both sides of the planar carrier, so that overall, more comprehensive information about the individual capillary tube can be provided on both sides. As a rule, providing information on one side is sufficient so that all information can be recorded from one direction of view onto the holder (cf. further below). For the electronic recording of information both sides of the carrier can of course be used. The holder according to the invention works like a small, lightweight grip for the individual capillary tube. Its dimensions are of the order of the capillary tube and can be of the size of a normal paper clip. Through the direct markability of the sample on the entire holder, a further step is taken towards the standardisation and digitalisation of sample referencing in line with the DFG guidelines of quality-assuring scientific practices.

Of importance for the safe handling of the extremely breakable capillary tube is its secure holding in the carrier. This can already been achieved through clamping jaws, whereby, however, the clamping force is limited by the fragility. According to the invention, the carrier is thinner than the capillary tube to be held. Hence, a held capillary tube projects slightly with its wall laterally on both areas of the carrier. In a first embodiment of the holder, it is preferably and advantageously envisaged that an inscribed label or adhesive strip is stuck on the marking field. In this way, the capillary tube is additional securely fixed by adhesion on the carrier in the area of the arms. However, the adhesion is reversible, and after removal of the label or adhesive strip (on one or both sides of the carrier), the capillary tube can be easily pulled out of the clamping without adhesive residues. The holder and capillary tube are thus reusable. The label or adhesive strip can be inscribed and contain information about the contents of the tube. The label can, for example, be a self-adhesive inscription strip than can be written on by hand or with an inscribing machine. The adhesive strip can be a transparent adhesive strip. It can, of course, be inscribed by hand or have an inscribed label stuck over it.

In accordance with a next embodiment of the invention, it is particularly preferable and advantageous if the carrier is rectangular, wherein the slit is arranged centrally in the carrier. This results in a rectangular flag with an elongated slit on one half. A rectangular slit also produces rectangular arms. Such an embodiment is particularly suitable for the insertion, holding and handling of a capillary tube and also for simply manufacturability of the carrier.

In a next embodiment of the invention, it is envisaged that the arms have grooves on their opposite inner sides, wherein the width of the grooves is matched to the diameter of the capillary rods to be held. Through this measure the test tube can, so to speak, be “locked into place” in the holder. It is precisely positioned in the holder and cannot be twisted out of the holder along its longitudinal axis. In addition, it can also be preferably and advantageously envisaged that the slit is trapezoidal, wherein it is tapered towards one end of the carrier. In this way the arms, and therefore the clamping jaws, converge slightly so that defined pre-tensioning for clamping can be provided. The capillary tube can thus be securely clamped in the holder without the risk of breaking or sliding out again. Nevertheless, it is just held by clamping so that it can be removed from the holder. It can then be reused after cleaning, for example, which is particularly advantageous. Furthermore, preferably and advantageously the slit can widen at its end facing away from an end of the carrier. A rectangular recess, for example, is produced at the open end of a received capillary tube. This ensures that the end is not accidentally closed. However, in the recess there is space for a small closure cap or wax stopper if the capillary tube is to be closed, in the event of longer storage, for example. At the same time, the main arm of the recess as a height stop for the capillary tube when it is pushed into the holder. This ensures that when several holders are used, the capillary tube exhibits the same free length and can be consistently placed in a measuring device.

In a next embodiment of the holder according to the invention, it is preferably and advantageously envisaged that the arms at one end of the carrier each have a bevel on their outer sides. These are for centring the holder when placing in, for example, a spinner or carrier block. Furthermore, the holder is well-matched to the capillary tube. There are no sharp edges on the carrier which could result in breakage of an inserted capillary tube. In addition, the entire holder can have a circumferential chamfer that facilitates manufacturing and handling.

Possible uses of the holder have been listed above. A preferred use is the insertion of a capillary tube into a spinner, which in turn can be inserted into the examination chamber of an NMR spectrometer (Nuclear Magnetic Resonance). It is thus advantageous if the holder's dimensions are such that it can be inserted into a receiving opening of a spinner. In doing so, through the provision of bevels on the outer arm ends, the holder can centre itself in a simple and completely adequate manner. In measuring operation, the carrier with the capillary tubes is suspended freely in the spinner and lifted slightly by an air flow. The spinner rotates relatively slowly so that the holder also rotates through air friction. The capillary tube can remain in the holder for a long time and be easily transported, used and stored therein. Here, it is largely protected against breakage. But after use it can also be clipped back out of the holder and then disposed of or cleaned. It is the available for re-use, which is particularly environmentally-friendly. The carrier c is also suitable for use in all other applications in which capillary tubes are used (including outside of measuring apparatuses, for example during filling, transportation or storage) as it constitutes a simple, practical handling device for the capillary tube.

The holder is configured like a planar flag and only has a central slit on one side. It is particularly simple to manufacture. It applies even more so if, in accordance with a further embodiment of the invention, the carrier is made of a plastic that can be processed with 3D printing. The holder can then be easily and cost-effectively manufactured as a mass product-similar to a plastic paper clip. Coloured plastics in different colours can also be used for this. In addition, different sizes of the holder, for example the length of the two arms, can be easily produced, and, more particularly, the holder can be easily adapted to differently sized capillary tubes or other surrounding conditions, for example the spinner size.

The holder fulfils two main functions: it allows secure, comprehensive handling and clear marking of a capillary tube. A third function can be considered as the simple and completely adequate self-centring in a spinner or collection device, wherein through self-centring all marking fields on the holder can easily be seen at the same time. Secure handling not only includes using the capillary tube in measuring operations, but also its secure storage outside of measuring operations. According to the invention, a collection device a plurality of holders can be provided, in which arranged in one carrier block are several receiving slits with through holes, wherein the holders can be inserted into the slits and the capillary tubes to be held into the through holes. In this way the capillary tubes can be arranged very clearly, if, preferably and advantageously, the receiving slits are arranged obliquely and in several parallel rows. The information applied to the carriers can then be clearly read, so that finding sought capillary tubes is particularly easy. Moreover, at least the carrier block can preferably and advantageously be produced through 3D printing from a (coloured) plastic. Also preferably and advantageously, a cover hood can be provided which protects the inserted capillary tubes from dirt and damage. The carrier block can be stored in a box, so that simple storage and handling of capillary tubes clipped into the carrier is possible. The box and cover hood can be identical components, made of transparent plastic, for example. Further explanations of the present invention and its mentioned modifications, are set out in the following example of embodiment.

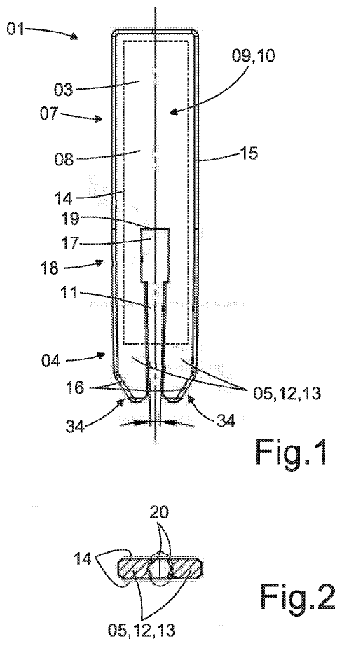

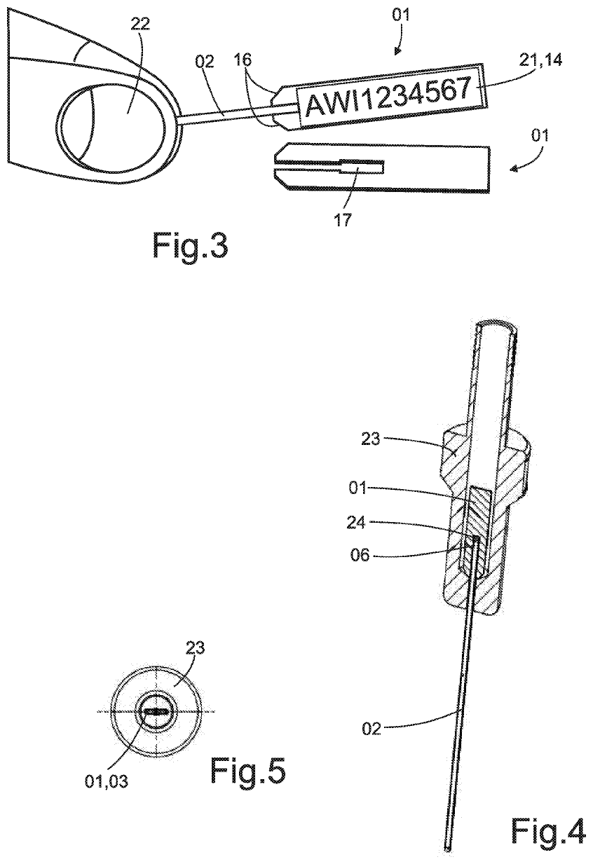

FIG. 1 shows a holder 01 for a capillary tube 02 (cf. FIG. 4). The holder 01 consists of an elongated carrier 03 which at its one end 04 has two clamping jaws 05 at a distance from one another. These are for receiving one end 08 (the “filling end”) of the capillary tube 02. At its other end 07, the carrier 03 has a handle 08, wherein this is connected in one piece to the carrier 03, i.e., is implicitly part of the carrier 03. The handle 08 has an elongated area 09. In the present invention this is exclusively in the form of a planar area 10—the prior art has crossed or curved areas here, which are poorly suited for the application of markings. At its one end 04, the carrier 03 has an elongated slit 11, through which two opposite arms 12, 13 are formed in the carrier 03 which act as clamping jaws 05. In the shown example of embodiment, the slit 11 is arranged centrally in the carrier 03 so that the two arms 12, 13 have the same width and are symmetrical. The planar area 09 of the handle 08 is designed as a marking field 14 (shown by the dashed line) for the easy application of information, cf. FIG. 3.

In the example of embodiment shown in FIG. 1, the carrier is 03 is approximately rectangular. It can, however, be of any other expedient shape, for example, triangular, wherein the handle 08 is wider than the arms 12, 13 or with a disk-like handle 08 on elongated arms 12, 13. As the holder 01 can, in particular, be made by means of 3D printing (additive manufacturing) from a (coloured) plastic, the embodiment possibilities are only limited functionally by use and manufacturing.

In FIG. 1 it is also shown that the slit 11 is slightly trapezoidal (3.6° opening angle, indicated in FIG. 1 by the angle sign), through which the carrier 03 tapers slightly toward its one end 04. As a result, the arms 12, 13 or the clamping jaw 05 converge slightly so that the capillary tube 02 can be clamped in with defined pre-tensioning. It can also be seen in FIG. 1, that the carrier 03 has a circumferential chamfer 15 which simplifies the manufacturing process. On their outer sides 34, the two arms 12, 13 each have a bevel 15 which provides continuous transition to the capillary tube 02 that prevents breakage. Furthermore, on insertion into a spinner 23 (cf. FIG. 4) or into a collection device 25 (cf. FIG. 5), via the bevels 15 effective self-centring of the holder 01 and thereby the capillary tube 02 takes place. Particularly when placing several capillary tubes 02 in the collection device 25, this self-centring ensures that the marking fields 14 of all capillary tubes 02 are oriented in one line of sight and can be read (out) simultaneously by the user.

In addition, in FIG. 1 a widening 17 is shown on the slit 11 which is arranged at its end 19 that faces away from an end 04 of the carrier 03. The widening 17 acts as a main bore. It can receive the open end of the capillary tube 02 so that it is not accidentally closed. It can, however, also receive closures of the capillary tube 02, cf. FIG. 4. At the same time, a main arm 19 of the widening 17 acts as a height stop on inserting the capillary tube 02 into the holder 01.

FIG. 2 show the holder 01 according FIG. 1 in cross-section. Shown are the two arms 12, 13 which on their opposite inner sides have grooves 20 and form the clamping jaws 05. The width of the grooves 20 is matched to the diameter (show by the dashed line in FIG. 2) of the capillary tube 02 to be held so that it can be securely held between the clamping jaws 05. The capillary tube 02 is, so to speak, clipped into the holder 01. The carrier 03 can also be smaller in thickness than the diameter of the capillary tube 02 to be held. Through this is projects laterally slightly and can be fixed to the carrier 03 with adhesive tape. In the shown example of embodiment, the marking field 14 is provided on both sides of the carrier 03. The capillary tube 02 can therefore be fixed to both sides of the carrier 03.

FIG. 3 shows a capillary tube 02 in a holder 19 with an applied label 21 on the marking field 14. The indicated thumb 22 shows how large the marking field 14 on the holder 01 according to the invention is, so that more comprehensive information (also handwritten, which usually required more space than printed information) about the sample in the capillary tube 02 can be provided. As the label 21 can also be stuck over the widening 17 (cf. additionally shown holder 01), the marking field 14 still increases, thereby taking up almost the entire carrier 03, possibly even on both sides. At the same time, beneath the label 21 the bevels 16 can also be seen in the area of the clamping jaws 05, that fit the carrier 03 well to the capillary tube 02 and serve to centre the holder 01.

FIG. 4 shows a spinner 23 of an NMR spectrometer into which a capillary tube 02 together with the correspondingly dimensioned holder 01 has been inserted. The spinner 23 is a rotor as part of an electric motor, through which the capillary tube 02 is rotated during measurement. In the shown example of embodiment, the capillary tube 02 is closed with a wax stopper 24. FIG. 5 shows a cross-section through the spinner 23 with an inserted 01. It can be seen how the planar carrier 03 fits in well. Jamming as in the case of leading round carriers or stoppers in the spinner 23 is avoided.

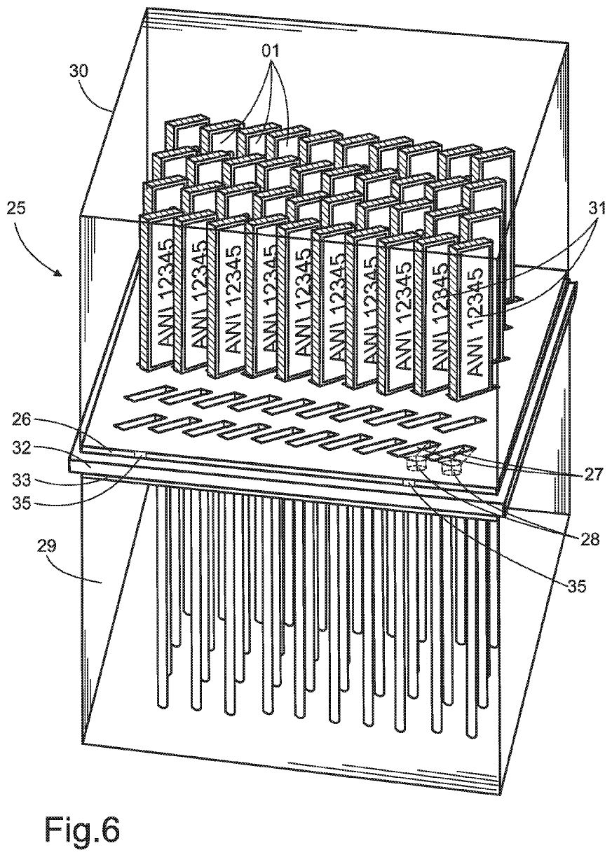

FIG. 6 show a collecting device 25 for a plurality of holders 01 according to the invention. In a carrier block 26, which can also be manufactured by way of 3D printing from a (coloured) plastic, there is a plurality of receiving slits 27 which are matched to the cross-sectional dimensions of the carrier 03. Provided in the base of the receiving slits 27 are through holes 28 (indicated by the dashed line in FIG. 6) matched to the geometry of the capillary tube 02 clipped into the holder 01, through which the capillary tubes 02 clipped into the holder 01 are pushed. In the shown example of embodiment, the receiving slits 27 are arranged in several rows and columns in oblique alignment, so that all marking fields 14 of the inserted holders 01 are clearly visible and these can easily be removed. Other expedient arrangements of the receiving slits 27 are of course also possible. In the shown example of embodiment, every receiving slit 27 is bevelled towards the through hole 28 (also indicated by the dashed line in FIG. 6) so that on insertion the capillary tube 02 is well-centred and cannot break off. The collecting device 25 with the inserted holders 01 and capillary tubes 02 is stored in a box 29. This is covered with a cover hood 30 so that the marking fields 14 and/or applied information are protected against environmental influences. In the shown example of embodiment, the box 29 and the cover hood 30 are identical components made of transparent plastic, which fulfil both functions (standing and covering). The carrier block 26 is applied to a frame 32 which on its underside has a projection 33 for pushing on the box 29. The cover hood 30 is pushed and clamped fast via lugs 35 on the carrier block 26.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B.” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE NUMBERS

01 Holder
02 Capillary tube
03 Carrier
04 One end of 03
05 Clamping jaws
06 One end of 02 (open filling end)
07 Other end of 03
08 Handle
09 Elongated area on 08
10 Planar area on 08
11 Slit in 03
12 One arm
13 Other arm
14 Marking field
15 Circumferential chamfer
16 Bevel
17 Widening
18 one end of 11
19 Main arm of 17
20 Groove
21 Label
22 Thumb
23 Spinner
24 Wax stopper
25 Collecting device
26 Carrier block
27 Receiving slit
28 Through hole
29 Box
30 Cover hood
31 Information
32 Frame
33 Projection
34 Outer side 12, 13
35 Lug on 26

1. A holder for a single capillary tube, comprising:
an elongated carrier which at one end thereof has two clamping jaws at a distance from each other configured to receive one end of the capillary tube and an other end thereof a handle with an elongated planar area, the handle being connected to the elongated carrier in one piece,
wherein
the elongated carrier comprises a planar area and is thinner than a diameter of a capillary tube to be held, and at one end the elongated carrier has an elongated slit forming two opposite arms comprising clamping jaws,
wherein a planar area of the handle comprises a marking field configured for applying information, and
wherein the marking field is arranged on one side or on both sides of the elongated carrier.

2. The holder of claim 1,
wherein
an inscribed label or an adhesive strip is stuck onto the marking field.

3. The holder of claim 1,
wherein
the elongated carrier is rectangular, and
wherein the slit is arranged centrally in the carrier.

4. The holder of claim 1,
wherein
on opposite inner sides thereof, the arms have grooves, and
wherein a width of the grooves is matched to a diameter of the capillary tube to be held.

5. The holder of claim 1,
wherein
the slit is trapezoidal, and
wherein the slit tapers toward one end of the elongated carrier.

6. The holder of claim 1,
wherein
at an end thereof facing away from one end of the elongated carrier, the slit has a widening with a main arm which acts as a height stop for the capillary tube to be held.

7. The holder of claim 1,
wherein
the arms on one end of the elongated carrier each have a bevel on their outer sides thereof.

8. The holder of claim 1,
wherein
dimensions of the holder are such that the holder is insertable into a receiving opening of a spinner of a spectrometer.

9. The holder of claim 1,
wherein
the carrier comprises a plastic that is processable with 3D printing.

10. A collecting device for a plurality of the holder of claim 1, comprising:
a carrier block having arranged therein a plurality of receiving slits with through holes,
wherein the holders are insertable into the slits and the capillary tubes to be held are placeable in the through openings.

11. The collecting device of claim 10,
wherein
the receiving slits are oblique and arranged in several parallel rows.

12. The collecting device of claim 10,
wherein
the carrier block is storable in a box and/or covered with a cover hood.

13. The collecting device of claim 10,
wherein
at least the carrier block comprises a plastic that is processable with 3D printing.
